(19) United States
(12) Patent Application Publication
(10) Pub. No.: US 2022/0107299 A1
(43) Pub. Date: Apr. 7, 2022

(54) INFORMATION PROCESSING APPARATUS FOR VISUALIZING COLLECTED DATA BY GRAPH, CONTROL METHOD THEREOF, AND STORAGE MEDIUM

(71) Applicant: CANON KABUSHIKI KAISHA, Tokyo (JP)

(72) Inventor: Kohei Iwabuchi, Kanagawa (JP)

(21) Appl. No.: 17/555,031

(22) Filed: Dec. 17, 2021

Related U.S. Application Data
(63) Continuation of application No. PCT/JP2020/022323, filed on Jun. 5, 2020.

(30) Foreign Application Priority Data
Jun. 27, 2019 (JP) 2019-120328

(51) Int. Cl. G01N 33/00; A01G 7/00; G01D 7/02; G01N 33/02

(52) U.S. Cl. CPC G01N 33/0098; G01N 33/025; G01D 7/02; A01G 7/00

(57) ABSTRACT

An information processing apparatus configured to display results of measurement for a plurality of items about an agricultural crop as a graph, acquires measurement information including an item for which the measurement is performed and a measured value obtained by the measurement, acquires information about a reference value set for each of the plurality of items, performs conversion of the measured value into a display coordinate value on a graph for each of the plurality of items to make reference values agree with each other on the graph, and displays a graph including a plurality of pieces of series data corresponding to the respective plurality of items and having display coordinate values converted by the conversion unit, and an index indicating the reference values for the plurality of items.

### CROSS-REFERENCE TO RELATED APPLICATIONS

This application is a Continuation of International Patent Application No. PCT/JP2020/022323, filed Jun. 5, 2020, which claims the benefit of Japanese Patent Application No. 2019-120328, filed Jun. 27, 2019, both of which are hereby incorporated by reference herein in their entirety.

### BACKGROUND OF THE INVENTION

### Field of the Invention

The present invention relates to a technique for visualizing collected data by a graph.

### Background Art

Conventionally, visualization of collected numerical data in graph form has been performed. Japanese Patent Application Laid-Open No. 11-283042 discusses a method in which a plurality of pieces of data each having a normal range defined by an upper limit and a lower limit is arranged in one graph, and the widths and the positions of the normal ranges of the respective data are matched with each other. According to Japanese Patent Application Laid-Open No. 11-283042, even if a plurality of series of data is simultaneously displayed, it is easy to intuitively understand whether each fits into the normal range.

The purposes of data collection vary. For example, there is a case where the purpose is to check how data collected by measuring numerical values of items about a predetermined object approaches a reference value over time, or to predict a timing at which the data reaches the reference value. In a case where a measured value reaching the reference value is a positive phenomenon, the reference value can be referred to as a target value. Even if the widths and the positions of the plurality of series of data are aligned as in Japanese Patent Application Laid-Open No. 11-283042, it is still necessary to determine a relationship between each data and each reference value individually.

### CITATION LIST

### Patent Literature

PLT 1: Japanese Patent Application Laid-Open No. 11-283042

### SUMMARY OF THE INVENTION

According to an aspect of the present invention, an information processing apparatus configured to display results of measurement for a plurality of items about an agricultural crop as a graph, includes a measurement information acquisition unit configured to acquire measurement information including an item for which the measurement is performed and a measured value obtained by the measurement, a reference information acquisition unit configured to acquire information about a reference value set for each of the plurality of items, as a value to be compared with the measured value that changes over time; a conversion unit configured to perform conversion of the measured value into a display coordinate value on a graph for each of the plurality of items based on the measurement information and the reference value, to make reference values set for the respective plurality of items agree with each other on the graph; and a display control unit configured to execute control for displaying a graph including a plurality of pieces of series data corresponding to the respective plurality of items and having display coordinate values converted by the conversion unit, and an index indicating the reference values for the plurality of items.

Further features of the present invention will become apparent from the following description of exemplary embodiments with reference to the attached drawings.

### BRIEF DESCRIPTION OF THE DRAWINGS

FIG. 1A is a diagram illustrating an example of a hardware configuration of an information processing apparatus.

FIG. 1B is a diagram illustrating an example of a functional configuration of the information processing apparatus.

FIG. 2A is a diagram illustrating an example of an operation screen displayed by the information processing apparatus.

FIG. 2B is a diagram illustrating an example of an operation screen displayed by the information processing apparatus.

FIG. 3A is a diagram illustrating an example of a table for managing information predefined about growing of a crop.

FIG. 3B is a diagram illustrating an example of a table for managing information predefined about the growing of a crop.

FIG. 4A is a diagram illustrating an example of a table for managing reference information related to a plurality of measurement items.

FIG. 4B is a diagram illustrating an example of a table for managing measurement information related to a plurality of measurement items.

FIG. 5A is a diagram illustrating an example of a table used in measured-value conversion processing.

FIG. 5B is a diagram illustrating an example of a table used in the measured-value conversion processing.

FIG. 5C is a diagram illustrating an example of a table used in the measured-value conversion processing.

FIG. 6 is a flowchart illustrating graph generation processing.

FIG. 7 is a flowchart illustrating measured-value conversion processing.

FIG. 8A is a diagram illustrating an example of a graph displayed by the information processing apparatus.

FIG. 8B is a diagram illustrating an example of a graph displayed by the information processing apparatus.

FIG. 9A is a diagram illustrating an example of a graph displayed by the information processing apparatus.

FIG. 9B is a diagram illustrating an example of a graph displayed by the information processing apparatus.

FIG. 10 is a diagram illustrating an example of a graph displayed by the information processing apparatus.

FIG. 11A is a diagram illustrating an example of a graph displayed by the information processing apparatus.

FIG. 11B is a diagram illustrating an example of a graph displayed by the information processing apparatus.

FIG. 12 is a diagram illustrating an example of a table for managing reference information and measurement information related to a plurality of measurement items.

FIG. 13 is a diagram illustrating an example of a graph displayed by the information processing apparatus.

FIG. 14 is a flowchart illustrating graph generation processing.

FIG. 15 is a flowchart illustrating measured-value conversion processing.

FIG. 16A is a diagram illustrating an example of a graph displayed by the information processing apparatus.

FIG. 16B is a diagram illustrating an example of a graph displayed by the information processing apparatus.

FIG. 17 is a diagram illustrating an example of a graph displayed by the information processing apparatus.

### DESCRIPTION OF THE EMBODIMENTS

Exemplary embodiments of the present invention will be described in detail below with reference to the attached drawings. Configurations to be described in the following exemplary embodiments are merely examples, and the present invention is not limited to the configurations illustrated in the drawings.

In the field of agriculture, various types of measurement are performed of a crop (agricultural crop) being grown over a fixed period of time. For example, there are measurement items representing maturity of a crop, such as a sugar content and an acid level. For each of the measurement items, a target value is set. Further, a change in a measured value with respect to the target value is visualized in a line graph or the like, so that a decision about growing of the crop is made. For example, among the measurement items representing the maturity of the crop, the sugar content indicates an upward trend and the acid level indicates a downward trend as the crop matures. A harvest date for the crop can be determined by observing the change in the measured value with respect to the target value for each of the measurement items. For example, in a case where the crop is grapes for wine production, the items for performing measurement related to a degree of maturity of the crop include the sugar content (Brix), acid level (TA), hydrogen-ion concentration (pH), anthocyanin content, weight of a bunch of ripe grapes, and effective accumulated temperature (growing degree days (GDD)).

There are measurement items closely linked to each other, as described by the example of the sugar content and the acid level, and it is desirable that the items can be checked simultaneously in one graph. However, if a plurality of measurement items is arranged in one graph, the amount of information in the graph increases because the measurement items each have a target value and a measured value, and thus, it is difficult to grasp a change in the measured value with respect to the target value for each of the measurement items. Further, collected data can have a tendency to rise or a tendency to fall over time, depending on the item. Even if the tendencies of the plurality of measurement items in the graph are simultaneously displayed in a seemingly easy-to-compare manner, it may be desirable to check a change in the measured value with respect to the target value individually in a case where data indicating an upward tendency and data indicating a downward tendency are mixed in the graph. Concerning such an issue, an example in which a plurality of series of data is displayed in one graph in which the positions of target values are adjusted to one point will be described in a first exemplary embodiment.

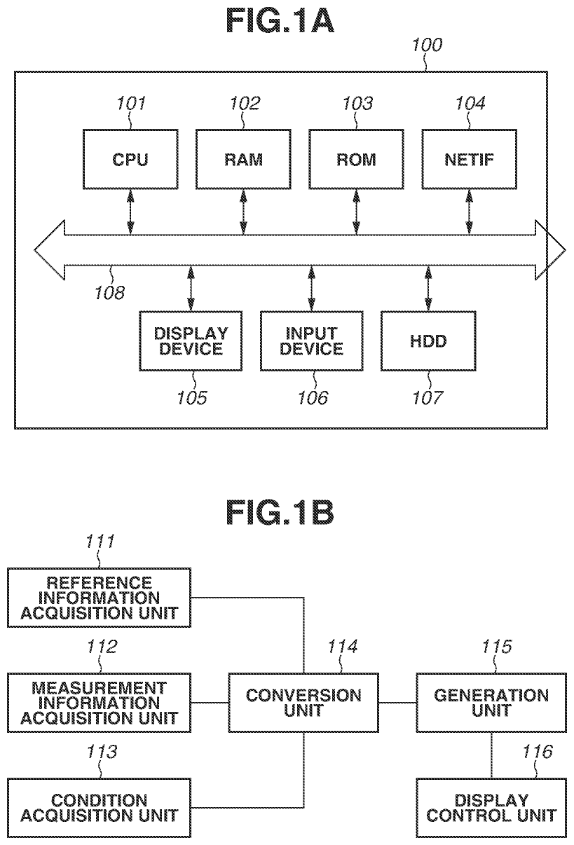

FIG. 1A illustrates an example of a diagram schematically illustrating a hardware configuration of an information processing apparatus 100 according to the first exemplary embodiment. A central processing unit (CPU) 101 is a central processing unit for controlling a computer system. The CPU 101 executes operations and processing of information, control of each hardware component, and the like, based on a control program and a processing program according to the present exemplary embodiment, so that various types of processing including graph generation processing to be described below and the configuration of each of functional blocks are implemented. A RAM 102 is a random access memory, and functions as a work memory used for loading of an execution program and execution of a program, as a main memory of the CPU 101. A ROM 103 is a read-only memory storing the control program defining an operation processing procedure for the CPU 101 and the processing program according to the present exemplary embodiment. The ROM 103 includes a program ROM storing an operating system (OS), which is a system program for control of devices of the computer system including various processing procedures to be described below, and a data ROM storing information necessary for the operation of the system. An HDD 107 to be described below can be used in place of the ROM 103.

A network interface (NETIF) 104 is a network interface and controls input and output of data transmitted and received via a network. The program according to the present exemplary embodiment may be downloaded via the NETIF 104 and stored in the HDD 107. A display device 105 is, for example, a cathode-ray tube (CRT) display or a liquid crystal display. An input device 106 is an operation input unit for accepting an operation instruction from a user, and examples thereof include a touch panel, a keyboard, and a mouse. The HDD 107 is a hard disk drive and is a storage device. The HDD 107 is used to store data including an application program. A bus 108 is an input-output bus (an address bus, a data bus, and a control bus) for connecting the above-described units.

FIG. 1B is a diagram illustrating functional blocks implementing the graph generation processing in the information processing apparatus 100 according to the present exemplary embodiment. The CPU 101 loads a program stored in the ROM 103 into the RAM 102 and executes the program, so that each of the functional blocks is implemented. Each of the functional blocks may be implemented by a hardware configuration, or may be implemented by a software configuration, or may be implemented by a combination of the hardware configuration and the software configuration. In a case where the hardware is configured, a computing unit or a circuit corresponding to the processing of each function unit to be described herein may be configured. The same holds true for functional blocks according to each of the exemplary embodiments to be described below.

A reference information acquisition unit 111 accepts information for setting a reference value for each of the measurement items. In the present exemplary embodiment, a “target value”, which is a value that each data is expected to approach or reach, is the reference value. However, the reference value is not limited to the “target value” as long as the reference value is a value to be compared with a measured value that can change over time. For example, there may be set a reference value that requires “controlling of the measured value not to approach too close to the reference value”, contrary to the target value, or “execution of predetermined work before the measured value reaches the reference value”. When the role (meaning) of such a reference value is common to a plurality of measurement items simultaneously visualized on a graph, it is easy for a user looking at the graph to understand the situation intuitively.

In the present exemplary embodiment, the reference information acquisition unit 111 acquires the value of the target value for each item freely input by the user via an operation screen displayed by the display device 105 and via the input device 106. The reference information acquisition unit 111 stores a correspondence relationship between the item and the target value in a predetermined form in a storage unit such as the HDD 107 or an external storage device connected via the NETIF 104. However, the target value may be acquired based on a notice from an external apparatus, or past results, without being input by the user directly operating the information processing apparatus 100. In addition, the target value may be changed or updated any time.

A measurement information acquisition unit 112 accepts information input to indicate a result concerning measurement performed one or more times for each of the measurement items. The information input to indicate the measurement result will be hereinafter referred to as “measurement information”. In the present exemplary embodiment, the measurement information is input by the user via an operation screen displayed by the display device 105 and via the input device 106. The measurement information includes at least a date and time when the measurement is performed, an item of the measurement, and information about a measured value.

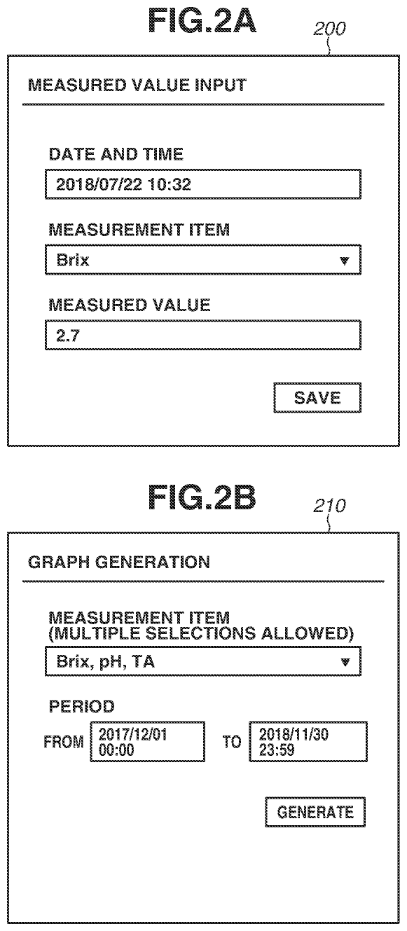

FIG. 2A illustrates an example of an operation screen displayed on the display device 105 to prompt the user to input the measurement result. In a screen 200, “Brix” representing one type of sugar content is selected as the measurement item. The user can input the measured value together with the date and time of the measurement of the sugar content, and give an instruction for saving. Each time the measurement information is obtained, the measurement information acquisition unit 112 stores the measurement information in a predetermined form in the storage unit such as the HDD 107 or the external storage device connected via the NETIF 104.

A condition acquisition unit 113 acquires conditions specifying an item and a period to be visualized as a graph of data collected by the measurement for the plurality of items. The item and the period to be visualized as the graph can be said to be conditions for selecting data for graphing, and thus will be hereinafter referred to as “selection conditions”. In the present exemplary embodiment, selection conditions input by the user via an operation screen displayed by the display device 105 and via the input device 106 are acquired as the selection conditions.

FIG. 2B is an example of an operation screen displayed on the display device 105 to prompt the user to input the selection conditions. In a screen 210, three items, the sugar content (Brix), pH, and acid level (TA), are selected as the measurement items. In this way, the plurality of items can be selected. In addition, in the screen 210, the user can freely designate the period by inputting a start date and time and an end date and time. The period may also be input by being selected from season information defined beforehand. In that case, the season information may be automatically selected based on the current date and time. The condition acquisition unit 113 stores the selection conditions in a predetermined form in the storage unit such as the HDD 107 or the external storage device connected via the NETIF 104.

A conversion unit 114 converts a measured value into a display coordinate value on a graph based on an item being a graphing target and the reference value (target value) corresponding to the item. In the present exemplary embodiment, one or more measured values related to the same item are extracted from the measurement information stored in the HDD 107 or the external storage device, and the extracted values are grouped based on the selection conditions acquired by the condition acquisition unit 113. Subsequently, conversion is performed by adding a common operation for each group. A generation unit 115 generates a graph in which the measured values converted by the conversion unit 114 are plotted. In the present exemplary embodiment, the generated graph is a line graph. However, the type of the graph is not limited to the line graph as long as the graph is in a form that can represent a change over time in the measured value in a manner that the change can be compared with the reference value (target value), for each of the measurement items.

The conversion unit 114 according to the present exemplary embodiment performs the conversion to make the reference values (target values) set for the respective plurality of items agree with each other on the vertical axis of the graph generated by the generation unit 115, as a result of the conversion for each item. The generation unit 115 displays an index for indicating a value corresponding to the target value on the vertical axis of the graph. Specific conversion processing and details of the graph will be described below. A display control unit 116 performs processing for generating and outputting image data for displaying the graph generated by the generation unit 115 on the display device 105 or an external apparatus connected via the NETIF 104.

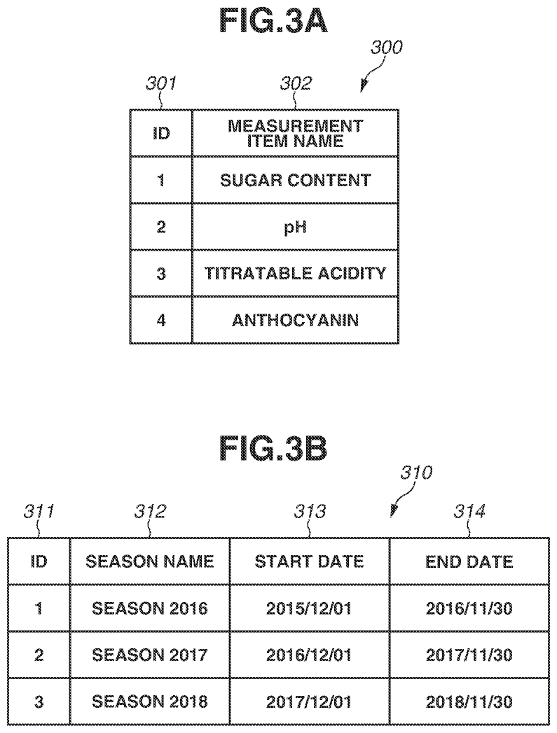

FIGS. 3A to 5C are diagrams illustrating examples of tables that indicate structures of various data used in the present exemplary embodiment. Each of the tables is stored in the HDD 107 or the external storage device, and is referred to by the function units of the CPU 101 in various processing procedures to be described below.

FIG. 3A is a diagram illustrating a measurement item table 300 used to manage the measurement items of one or more types of measurement performed with regard to growing of the crop. In an identifier (ID) 301, an ID for uniquely identifying a record in the measurement item table 300 is described. In a measurement item name 302, the name of the measurement item indicated by the corresponding ID is described. In this way, in the present exemplary embodiment, the measurement item is defined by a combination of the identifier and the name The name described in the measurement item name 302 can be used as a character string for display indicating the measurement item in a screen displayed on the display device 105.

FIG. 3B is a diagram illustrating a season information table 310 used to manage the season information. In the present exemplary embodiment, a season means a periodic length of time related to the growing of the crop. For example, one year may be one season. A period defined as one season is defined depending on the crop being grown. For example, in a field for growing grapes to be used for wine production, one year corresponds to one season, and each year can be referred to as a vintage. For a crop grown by double cropping, the season may be defined as a half year.

In an ID 311, an ID for uniquely identifying a record in the season information table 310 is described. In a season name 312, the name of the season information is described. The season name 312 can be used as a character string for display in a screen displayed on the display device 105 when information representing a period is to be selected from a list of the season information. In a start date 313, the start date of the season is described. In an end date 314, the end date of the season is described.

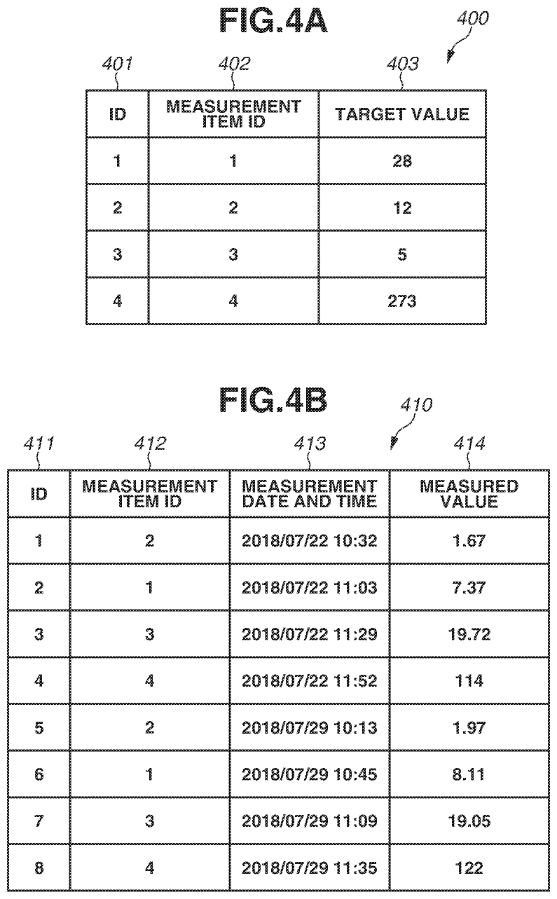

FIG. 4A is a diagram illustrating a target value table 400 for managing the information acquired by the reference information acquisition unit 111. In an ID 401, an ID for uniquely identifying a record in the target value table 400 is described. In a measurement item ID 402, a measurement item ID to indicate which measurement item a target value is associated with is described. In a target value 403, the target value of the measurement item identified by the measurement item ID 402 is described. In the present exemplary embodiment, the target value for each of the measurement items acquired by the reference information acquisition unit 111 is stored in the target value 403.

FIG. 4B is a diagram illustrating a measured value table 410 for managing the information acquired by the measurement information acquisition unit 112. In an ID 411, an ID for uniquely identifying a record value in the measured value table 410 is described. In a measurement item ID 412, a measurement item ID to indicate which measurement item a measured value is associated with is described. In a measurement date and time 413, the date and time when the measurement is performed is described. In a measured value 414, a measured value obtained by the measurement is described. In the present exemplary embodiment, the measured value acquired by the measurement information acquisition unit 112 is stored in the measured value 414.

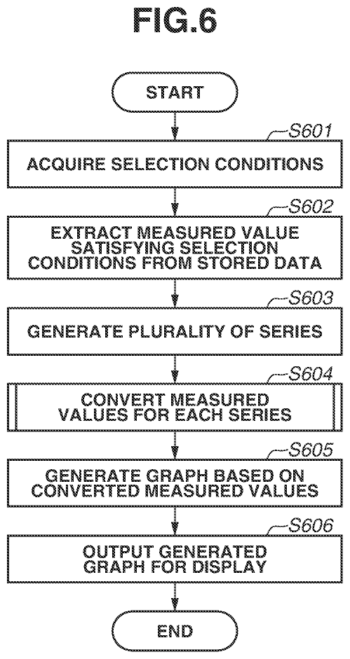

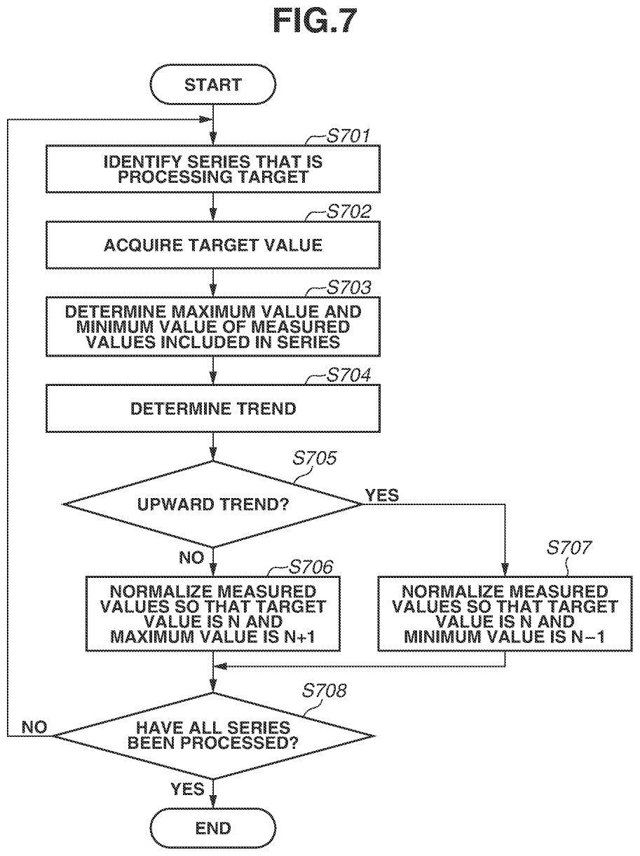

FIG. 6 and FIG. 7 are flowcharts illustrating the graph generation processing executed by the information processing apparatus 100. Each process (step) in the flowcharts is provided with an S at the top of each of the reference numerals thereof and will be described below.

FIG. 6 is a flowchart illustrating an example of main processing by the information processing apparatus 100 according to the present exemplary embodiment. In the present exemplary embodiment, the processing of the flowchart in FIG. 6 starts when an instruction for generating a graph is provided by the user by operating the screen 210. First, in step S601, the condition acquisition unit 113 acquires the selection conditions specified by the user.

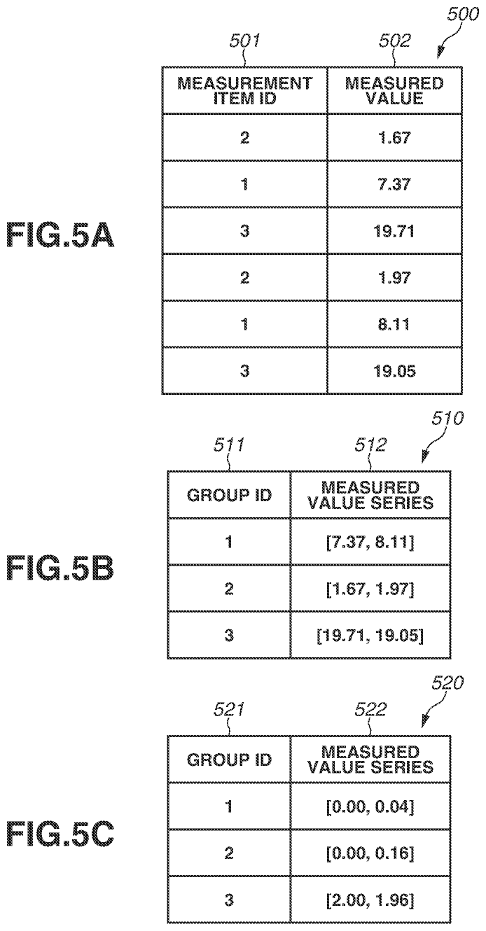

In step S602, the conversion unit 114 identifies a plurality of records satisfying the selection conditions acquired in step S601 in the measured value table 410 for managing the information acquired by the measurement information acquisition unit 112. The measurement item ID 412 and the measured value 414 are extracted from each of the identified records, and a piece of intermediate data consisting of the two extracted items is generated. FIG. 5A is a diagram illustrating an intermediate data table 500 used to hold a plurality of pieces of intermediate data generated in step S602. A measurement item ID 501 is the measurement item ID included in the intermediate data. A measured value 502 is the measured value included in the intermediate data.

In step S603, the conversion unit 114 generates series data for each item based on the plurality of pieces of intermediate data generated in step S602. In the present exemplary embodiment, the conversion unit 114 generates one series data by extracting one or more measured values related to the same item from the intermediate data table 500 and grouping the extracted measured values. The conversion unit 114 generates a plurality of pieces of series data by similarly performing grouping for each of the plurality of items. FIG. 5B is a diagram illustrating a measured value group table 510 used to hold groups of the measured values (the series data) generated in step S603. A group ID 511 is an ID for uniquely identifying the group. In the case of the present exemplary embodiment, the measurement item ID to be a key to search for the measured values for grouping is the group 1D. A measured value series 512 is the plurality of measured values included in the group. The measured value group table 510 illustrates the series data obtained before measured-value conversion processing in step S604 to be described below is performed.

In step S604, the conversion unit 114 performs processing for executing an operation for each series and converting the measured values into display coordinate values on the graph. The contents of the specific processing in step S604 will be described below with reference to the flowchart in FIG. 7. FIG. 5C illustrates a measured value group table 520 holding the series data after the measured-value conversion processing in step S604 is performed. A group ID 521 is an ID for uniquely identifying the group as with the group ID 511 of the measured value group table 510. A measured value series 522 is the plurality of measured values included in the group. In the case of FIG. 5C, the display coordinate value, which is the result of conversion of the measured value stored in the measured value series 512 by the conversion unit 114, is stored in the measured value series 522.

In step S605, the generation unit 115 generates a graph using all the series data included in the measured value group table 520 after the conversion processing. In a case where a line graph is generated, one series data is rendered as one continuous line. In the present exemplary embodiment, the horizontal axis of the line graph is a temporal axis indicating the time course. The vertical axis indicates a change in the measured value. The target value is explicitly indicated on the scale of the vertical axis. In step S606, the display control unit 116 generates image data for displaying the generated graph on the display device 105 and outputs the generated image data.

FIG. 7 is a flowchart illustrating the conversion processing for each item executed by the conversion unit 114 in step S604. In step S701, the conversion unit 114 identifies a series that is a processing target. Specifically, one group is selected with reference to the pre-conversion measured value group table 510. In step S702, the conversion unit 114 acquires a target value from the target value table 400 using the group ID 511 of the input measured value group as a key. In step S703, the conversion unit 114 determines each of a maximum value and a minimum value for the measured value series 512 of the input measured value group.

Then, in step S704, the conversion unit 114 determines the tendency (hereinafter referred to as the trend) of the change in the measured value for the measured value series 512 of the input measured value group. The tendency of the change in the measured value can be determined by approximating the measured value series by a straight line using a known technique such as the least-square method. Thus, the slope of the line obtained thereby may be used as the tendency of the change in the measured value. Alternatively, the trend may be defined beforehand for each item.

In step S705, the conversion unit 114 determines whether the series data identified in step S701 indicates an upward trend. If the series data identified in step S701 indicates an upward trend (YES in step S705), the processing proceeds to step S707. If the series data identified in step S701 indicates a downward trend (NO in step S705), the processing proceeds to step S706. In step S707, the conversion unit 114 performs processing of converting measured values included in the series data indicating the upward trend. In the case of the present exemplary embodiment, the measured values are normalized so that the target value is N and the minimum value is N−1 for the series that is the processing target. In step S706, the conversion unit 114 performs processing of converting measured values included in the series data indicating the downward trend. In the case of the present exemplary embodiment, the measured values are normalized so that the target value is N and the maximum value is N+1 for the series that is the processing target. N may be an integer greater than or equal to 1, but in the present exemplary embodiment, a case where N=1 will be described below. In other words, in the present exemplary embodiment, the measured values are normalized so that the maximum value after the conversion is 2 and the target value after the conversion is 1 in step S706, and the measured values are normalized so that the target value after the conversion is 1 and the minimum value after the conversion is 0 in step S707.

To be more specific, in step S706 and step S707 of the present exemplary embodiment, each measured value is converted using the following Equation 1. Equation 1 is a function that returns the post-conversion measured value (display coordinate value) using the measured value as an input.

$$\begin{matrix}[ Equation 1 ] & \\ {f}_{1} ( α ) = \{ \begin{matrix}( α - minimum ) / ( target - minimum ) ( trend = upward ) \\ 2 - ( maximum - α ) / ( maximum - target ) ( trend = downward )\end{matrix} & ( 1 )\end{matrix}$$

If N=1, the minimum value of the series having the upward trend is 0 and thus can be displayed at a position near the origin and the horizontal axis of the graph, so that it is easy for a person looking at the graph to make an analysis. However, N convenient for the purpose of the measurement may be set. In a case where N of the target value after the conversion is freely set, in addition to the conversion by Equation 1, shifting of the values of the entire graph may be performed without changing the positional relationship between the measured values and the target value.

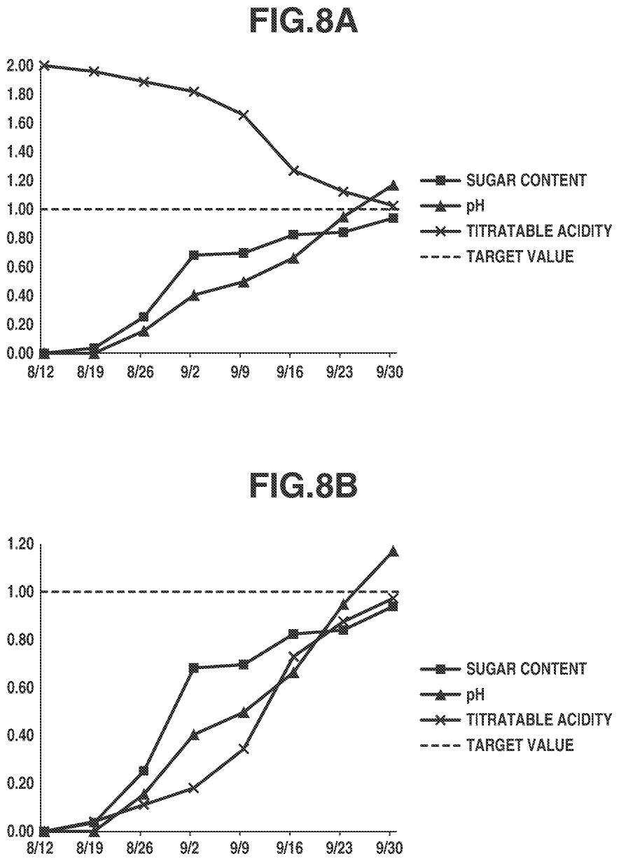

FIG. 8A and FIG. 8B are diagrams each illustrating the graph generated in step 5605. In the illustrated graph to be described below, the horizontal axis (an X-axis) represents the time, and the vertical axis (a Y-axis) represents the measured value. However, the values plotted on the vertical axis are the display coordinate values obtained by converting the measured values. FIG. 8A is a diagram illustrating the graph in a case where Equation 1 is used for the conversion of the measured values. In FIG. 8A, a plurality of pieces of series data corresponding to the plurality of measurement items are simultaneously displayed as line graphs. In FIG. 8A, the target values are converted into 1 for all the measurement items. Thus, in the vertical axis representing a range of the measured values, the index indicating the target values of all the measurement items is displayed at a position corresponding to 1 on the scale. A broken line indicating Y=1 is the index. The measured-value conversion processing in step S706 or step S707 is performed so that the measurement item on the upward trend is displayed to gradually approach the target value while rising, and the measurement item on the downward trend is displayed to gradually approach the target value while falling. This makes it easy for the user to grasp a relationship between the temporal change and the target value for the plurality of measurement items relating to each other.

Further, in the conversion of the measured values, transformation for adding processing of aligning the trends in a graph can be performed. FIG. 8B is an example of a graph in a case where all the same three series data as those in FIG. 8A are aligned to be on the upward trend. In this case, contents of the conversion processing in step S706 in the flowchart in FIG. 7 are changed. For example, the conversion is performed using the following Equation 2. As with Equation 1, Equation 2 is a function that returns the post-conversion measured value using the measured value as an input. However, Equation 2 is different from Equation 1 in that, for the measurement item on the downward trend, the measured values are normalized so that the maximum value after the conversion is 0 and the target value after the conversion is 1.

$$\begin{matrix}[ Equation 2 ] & \\ {f}_{2} ( α ) = \{ \begin{matrix}( α - minimum ) / ( target - minimum ) ( trend = upward ) \\ ( maximum - α ) / ( maximum - target ) ( trend = downward )\end{matrix} & ( 2 )\end{matrix}$$

The measurement item is displayed to gradually approach the target value while rising for both of the measurement item on the upward trend and the measurement item on the downward trend, by the transformation. In FIG. 8B, a used range of the vertical axis of the graph is narrow in comparison with that in FIG. 8A in which the measured value is converted by Equation 1. For this reason, a display area can be used widely in the vertical direction, and the granularity of numerical values displayed in the vertical axis can be made fine. Thus, the user can check the change in the measured value with fine granularity.

In addition, a configuration in which the pre-conversion measured value is presented in response to a user instruction input on the graph may be added as a different modification. In such a modification, the user can grasp the tendencies of the measured values relative to the target values for the plurality of measurement items at a glance and can specifically find the pre-conversion measured value at each plotted point. As the method of presenting the pre-conversion measured value to the user, two examples will be described below.

One of the examples is an example in which a change is added to the representation of the vertical axis. To be more specific, for each of the measurement items, a Y-axis representing a range of the measured value before the conversion is generated, and the Y-axis can be switched in response to an instruction input on the graph, and displayed. Because the range of the measured value before the conversion is indicated on the Y-axis of the graph, the user can grasp the pre-conversion measured value for each plotted point.

The size of the scale of the generated Y-axis is adjusted so that the target value and the maximum value or the minimum value for each of the measurement items agree with the target value and the maximum value or the minimum value of the measured value converted into the display coordinate value. In other words, in the case of the present exemplary embodiment, in a case where the measurement item indicates the upward trend, the Y-axis is generated in such a manner that the minimum value of the pre-conversion measured value is displayed at a position corresponding to 0 on the Y-axis of the graph, and the target value of the pre-conversion measured value is displayed at a position corresponding to 1. The scale may be graduated by performing interpolation (or extrapolation) linearly based on the position and the value of each of the minimum value and the target value. On the other hand, in a case where the measurement item indicates the downward trend, the Y-axis is generated in such a manner that the maximum value is displayed at a position corresponding to 2, and the target value is displayed at a position corresponding to 1. The scale may be graduated by performing interpolation (or extrapolation) linearly based on the position and the value of each of the maximum value and the target value.

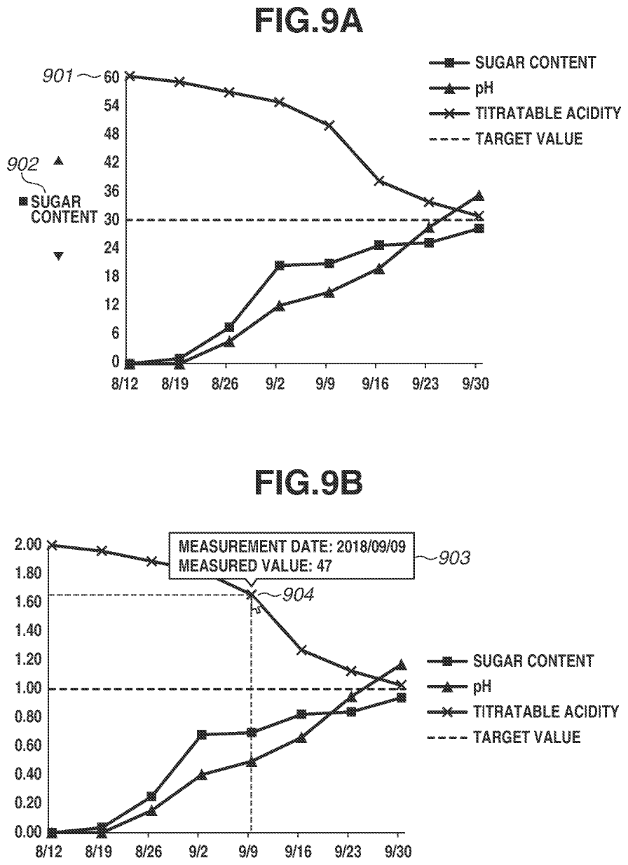

FIG. 9A is a diagram illustrating a graph in which the Y-axis representing the measurement value range before the conversion is displayed. FIG. 9A corresponds to a case where the vertical axis of the graph illustrated in FIG. 8A is changed into the Y-axis representing the pre-conversion measured value with regard to the sugar content among the three measurement items. An axis 901 is the Y-axis representing the measurement value range of the sugar content before the conversion. A selector 902 is a graphical user interface (GUI) component to be operated for the purpose of switching the Y-axis representing the measurement value range before the conversion in a case where the graph is generated for the plurality of measurement items. In FIG. 9A, the axis for the sugar content is displayed as an example, but the Y-axis representing the measurement value range before the conversion is similarly generated for other measurement items. The user can provide an instruction for switching the Y-axis by operating the selector 902. If the measured value series corresponding to the switched Y-axis among the plurality of measured value series rendered on the graph is highlighted when the Y-axis is switched by the selector 902, the relationship between the axis and the series data can be displayed in a more easily comprehensive way. The instruction for switching the axis is not limited to the method using the selector 902. For example, there may be provided a configuration in which, when any of the points plotted on the graph or any of the lines in the graph is designated by the input device 106, the axis is switched to the corresponding Ys

The other one of the examples is an example in which the pre-conversion measured value is displayed near the plotted point. To be more specific, in response to the designation of the point (measured value) plotted on the graph by the input device 106, the pre-conversion measured value corresponding to the designated point is displayed in a form of a tooltip or the like. The pre-conversion measured value can be determined using the following Equation 3 obtained by transforming Equation 1. Equation 3 is a function that returns the pre-conversion measured value using the post-conversion measured value as an input. However, in a case where the measured value is converted using Equation 2, a function for determining the pre-conversion measured value may be obtained by transforming Equation 2.

$$\begin{matrix}[ Equation 3 ] & \\ {f}_{3} ( β ) = \{ \begin{matrix}( target - minimum ) * β + minimum ( trend = upward ) \\ maximum - ( 2 * β ) ( maximum - target ) ( trend = downward )\end{matrix} & ( 3 )\end{matrix}$$

Alternatively, the measured value corresponding to the designated point may be obtainable without performing a reconversion by Equation 3. For example, it is possible to refer to the correspondence between the pre-conversion value and the post-conversion value by holding the series data based on the pre-conversion measured value in correspondence with the post-conversion measured value when performing the conversion processing in step S706 or step S707 of the flowchart in FIG. 7.

FIG. 9B is a diagram illustrating a graph having a function of displaying the pre-conversion measured value in a tooltip. A tooltip 903 is displayed in response to the designation of a point 904. The designation of the point 904 is, for example, input by hovering the mouse over the point 904. The tooltip 903 displays information about the measurement date and time and the pre-conversion measured value.

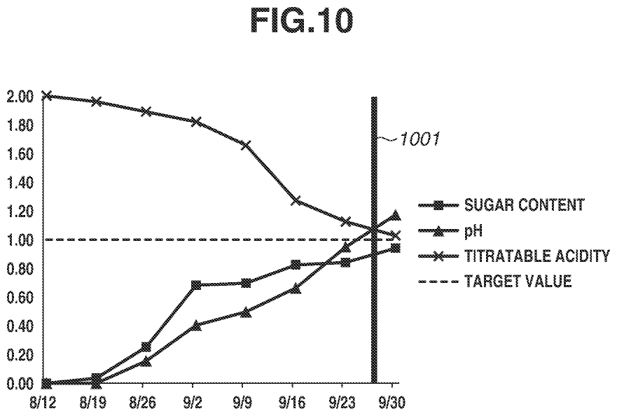

In another modification, a function of adding a new index to the horizontal axis representing the time course information of the graph can be provided. For example, in a case where the user confirming the relationship between the change of each of the series data on the growing of the crop such as grapes and the target value on the graph in FIG. 8A determines an expected harvest date, an index is added to a position corresponding to the date and time of the expected harvest date on the horizontal axis. Needless to say, a user interface (UI) element representing not only the expected harvest date of the crop but also any date such as a scheduled date of pesticide application can be displayed on the graph. FIG. 10 illustrates an example in which the modification is applied to the graph in FIG. 8A. A vertical line 1001 is an index representing the determined expected harvest date.

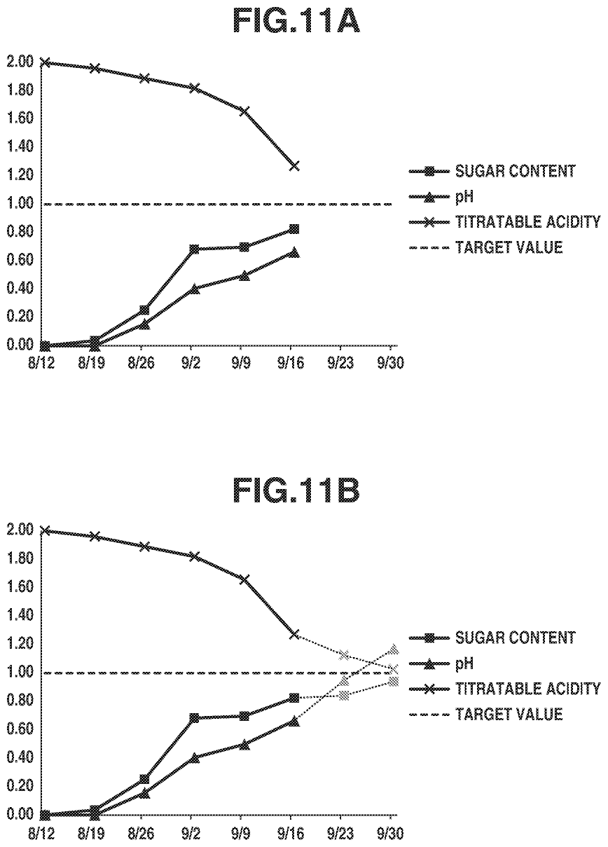

In yet another modification, a configuration may be provided for predicting a change in the measured value in the future based on a change in the measured value input in the past. FIG. 11A illustrates an example in which the modification is applied to the graph in FIG. 8A. In the graph in FIG. 11A, a blank space is provided so that a range of dates on the horizontal axis includes a specific date in an expected growing period of the crop irrespective of the current date and time. An example of the specific date may be a date expected to be the last date of the growing period. Since the blank space is provided, the user can easily predict how the measured value of each of the measurement items changes. For example, it is easy to predict the timing when the measured value of each of the measurement items reaches the target value. This assists the user in making a plan for performing a predetermined activity in appropriate timing in the future.

Further, FIG. 11B is a diagram illustrating a graph in which predicted measured values are plotted in the blank space in FIG. 11A as the measured values in the future. For example, the CPU 101 determines a function of an approximate curve by applying a known technique such as the least-square method to the past measured value series and inputs a future date to the function obtained thereby, so that the predicted measured value can be obtained. Further, the prediction of the measured value may be processed using a trained model obtained by machine learning. In that case, for example, the measured values of each of the measurement items for the past few years are prepared as training data, and knowledge is acquired from the data by machine learning. Subsequently, based on the acquired knowledge, a trained model is generated that obtains the measured values up to the middle of a season as input data and outputs the predicted values of the measured values to be obtained thereafter as output data. For example, the trained model can be a neural network model. Subsequently, the trained model performs the processing of a processing unit by operating in cooperation with a CPU, a graphics processing unit (GPU), or the like as a program for performing processing equivalent to the processing by the processing unit. The above-described trained model may be updated as appropriate after certain processing.

In this way, the information about the predicted measured value in the future is displayed, so that the user can grasp the relationship between the change in the predicted measured value and the target value as reference information. Thus, the user can easily make a plan for performing a predetermined activity in appropriate timing in the future.

As described above, in the first exemplary embodiment, in a case where a graph displaying measurement results for a plurality of items about a predetermined object is generated, the data for each of the items and the reference value (target value) therefor are aligned at the same position. Thus, the user can intuitively understand the relationship between the data for each of the items and the reference value therefor even if the plurality of series data is simultaneously displayed in the graph. In addition, in each of the above-described modifications, additional information about the decision making using the graph can be added to the graph. In this way, in the first exemplary embodiment and the modifications thereof, it is easy to collectively grasp the changes in the measured values with respect to the target values, and it is possible to effectively assist in making a decision using the graph.

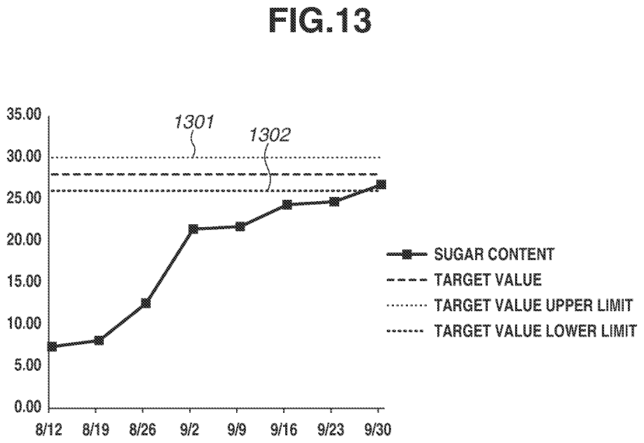

In the first exemplary embodiment, there is described the example applied to the case where, assuming that the reference value (target value) of each of the measurement items is a single value, the target values of all the measurement items are aligned at the same position when the graph is generated for the plurality of measurement items. A second exemplary embodiment to be described below is an exemplary embodiment suitable for a case where the reference value (target value) has a tolerance. In the second exemplary embodiment, when a graph is generated for a plurality of measurement items, the widths and the positions of tolerances of target values of the plurality of measurement items are aligned. Hereinafter, operation for aligning the widths and the positions of the tolerances will be described as “fitting of tolerance”. The definition of the tolerance will be described below with reference to FIG. 13.

The second exemplary embodiment is implemented by the information processing apparatus 100 having the hardware configuration illustrated in FIG. 1A and the functional configuration illustrated in FIG. 1B, as with the first exemplary embodiment. To avoid a repeated description, a detailed description will be omitted as appropriate for a part common to the first exemplary embodiment, and mainly a difference will be described. In the drawings, a part common to the first exemplary embodiment will be assigned the same reference numeral as that of the first exemplary embodiment.

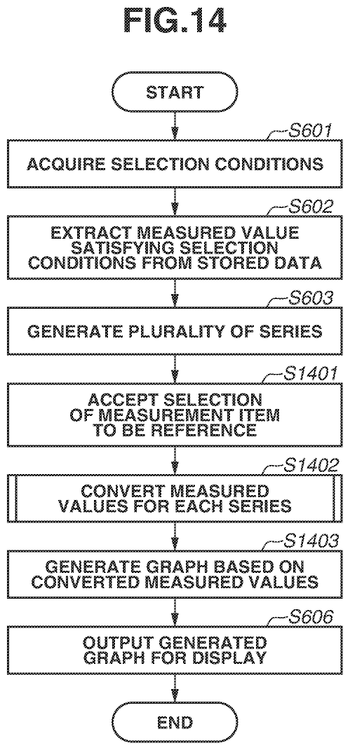

In the second exemplary embodiment, operations of the functional blocks are different from those of the first exemplary embodiment as follows. The reference information acquisition unit 111 according to the second exemplary embodiment acquires an allowable value in addition to the target value for each item freely input by the user. Subsequently, the correspondence relationship between the item and the target value and the allowable value is stored in a predetermined form in the storage unit such as the HDD 107 or the external storage device connected via the NETIF 104. The conversion unit 114 according to the second exemplary embodiment converts the measured value into the display coordinate value based on the target value, the allowable value, the measured value, and the selection conditions acquired by the reference information acquisition unit 111, the measurement information acquisition unit 112, and the condition acquisition unit 113. In the second exemplary embodiment, in particular, positions at which the target values of the plurality of measurement items are displayed on a graph are aligned, and fitting of the tolerance of the target value is performed. Details of the conversion of the measured value will be described below with reference to FIG. 14.

The generation unit 115 according to the second exemplary embodiment generates a graph from the measured values converted in the conversion unit 114. The target values and the tolerances of the respective measurement items agree with each other on the graph. The generated graph is assumed to be typically a line graph, but is not limited thereto as long as the graph is in a form that can represent a change in the measured value with respect to the target value of each of the measurement items.

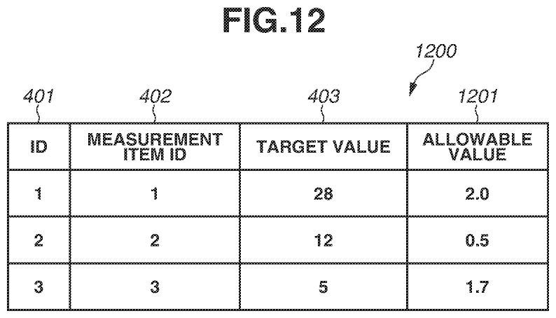

FIG. 12 is a diagram illustrating a target value table 1200 for managing the information acquired by the reference information acquisition unit 111. The target value table 1200 corresponds to the target value table 400 according to the first exemplary embodiment. However, in the target value table 1200, information about an allowable value 1201 is described in addition to the ID 401, the measurement item ID 402, and the target value 403. In the allowable value 1201, the allowable value of the measurement item identified by the measurement item ID 402 is described. In the present exemplary embodiment, the allowable value input together with the target value in correspondence with the measurement item is stored in the allowable value 1201 by the reference information acquisition unit 111.

FIG. 13 is a diagram illustrating an example of the tolerance of the target value on the graph. Here, a description will be provided using the series data of the sugar content as an example. An upper limit line 1301 is an object rendered on the graph to represent the upper limit of the tolerance of the target value. The upper limit of the tolerance of the target value here is a value obtained by adding a value stored in the allowable value 1201 to a value stored in the target value 403. A lower limit line 1302 is an object rendered on the graph to represent the lower limit of the tolerance of the target value. The lower limit of the tolerance of the target value is a value obtained by subtracting a value stored in the allowable value 1201 from a value stored in the target value 403. In FIG. 13, the upper limit line 1301 and the lower limit line 1302 are rendered to visualize a range between the lines on the graph as the tolerance of the target value.

FIG. 14 is a flowchart illustrating graph generation processing in the information processing apparatus 100 according to the second exemplary embodiment. A process similar to that in the flowchart in FIG. 6 described in the first exemplary embodiment is indicated by the same reference numeral as that in FIG. 6. Here, a point different from the first exemplary embodiment will be described.

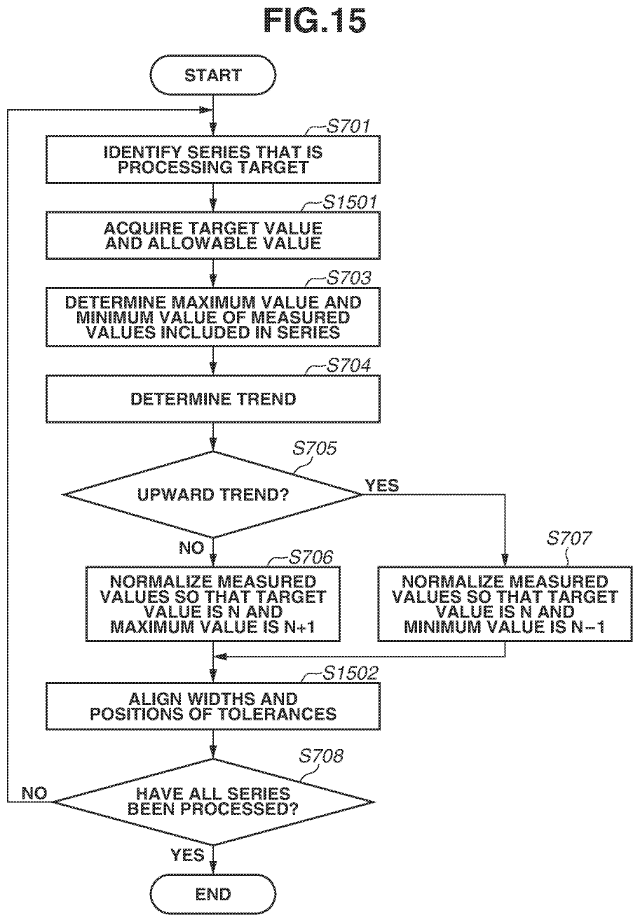

In step S1401, the conversion unit 114 accepts the input of a measurement item to be used as a reference in tolerance fitting processing to be described below, in response to a user operation. In the tolerance fitting processing (step S1502 to be described below), fitting of the tolerances of other measurement items is executed using the tolerance set for the selected measurement item as the reference. Details of conversion processing in the second exemplary embodiment executed in step S1402 will be described below with reference to a flowchart in FIG. 15.

In step S1403, the generation unit 115 generates a graph using all the series data included in the measured value group table 520 after the conversion processing. However, in the second exemplary embodiment, the tolerance of the measured value is explicitly displayed on the generated graph. In a case where a line graph is generated, one measured value group is rendered on a graph as one line. Further, the target value is rendered at a position corresponding to 1 on the scale on an axis representing a range of the converted measurement values. In the present exemplary embodiment, to render the upper limit line and the lower limit line on the graph, the generation unit 115 first acquires the target value and the allowable value from the target value table 1200 for the measurement item (series data) to be the reference selected in step S1401. Subsequently, the upper limit of the target value is determined by adding the target value and the allowable value together, and the lower limit of the target value is determined by subtracting the allowable value from the target value. Further, each of the upper limit and the lower limit is converted by Equation 1 or Equation 2, and based on the converted values, a line corresponding to each of the converted values is rendered on the graph.

FIG. 15 is a flowchart illustrating the details of step S1402 in the second exemplary embodiment. A process similar to that of the flowchart in FIG. 7 is indicated by the same reference numeral as that in FIG. 7. In the second exemplary embodiment, in step S1501, the conversion unit 114 acquires the target value and the allowable value from the target value table 1200 using the group ID 511 of the input measured value group as a key.

Subsequently, in the second exemplary embodiment, the conversion unit 114 performs the tolerance fitting processing using the following Equation 4 and Equation 5 in step S1502, after completing the conversion of the measured value data in step S706 or step S707. In other words, calculation processing is performed for aligning the widths and the positions of the tolerances to be visualized on the graph. First, the widths of the tolerances are aligned using Equation 4. Equation 4 is a function that returns the reconverted measured value by performing a second reconversion to align the width of the tolerance with those of other measurement items using the measured value converted by Equation 1 or Equation 2 as an input. In Equation 4, a value obtained by converting the allowable value of the measurement item selected in step S1401 using Equation 1 or Equation 2 is used as an “allowable value of a measurement item to be a reference”. A value obtained by converting the allowable value acquired in step S1501 using Equation 1 or Equation 2 is used as an “allowable value” in Equation 4.

f₄(β)=β* (allowable value of a measurement item to be a reference/allowable value) (4)

Next, the positions of the tolerances are aligned using Equation 5. Equation 5 is a function that returns the measured value converted to align the positions of the tolerances of the respective measurement items, using the measured value converted by Equation 4 as an input. A value obtained by converting the target value of the measurement item selected in step S1501 using Equation 1 or Equation 2 is used for the target value of the measurement item to be the reference in Equation 5. A value obtained by converting the target value acquired in step S1501 using Equation 1 or Equation 2 is used for the target value in Equation 5.

f₅(γ)=γ+(target value of the measurement item to be the reference−f₄(target value) (5)

As with the first exemplary embodiment, transformation may be added to enable checking of the value of the pre-conversion measured value on the graph in the second exemplary embodiment. Specifically, in a manner similar to that described in the first exemplary embodiment, the Y-axis (vertical axis) of the graph can be switched to the Y-axis that can display the pre-conversion value of each of the measurement items, and displayed. The method of generating the Y-axis corresponding to each of the measurement items is similar to that of the first exemplary embodiment, and thus the description thereof will be omitted.

A tooltip that displays information about the pre-conversion measured value may be displayed in response to an operation on a point plotted on the graph in the second exemplary embodiment as well. The pre-conversion measured value is determined by reversely converting the post-conversion measured value using each of Equation 5, Equation 4, and Equation 1 (Equation 2 in a case where the conversion has been performed using Equation 2) in this order. A function to be used in the reverse conversion can be obtained by transforming each of Equation 1 (Equation 2 in the case where the conversion has been performed using Equation 2) for a, Equation 4 for 13, and Equation 5 for γ.

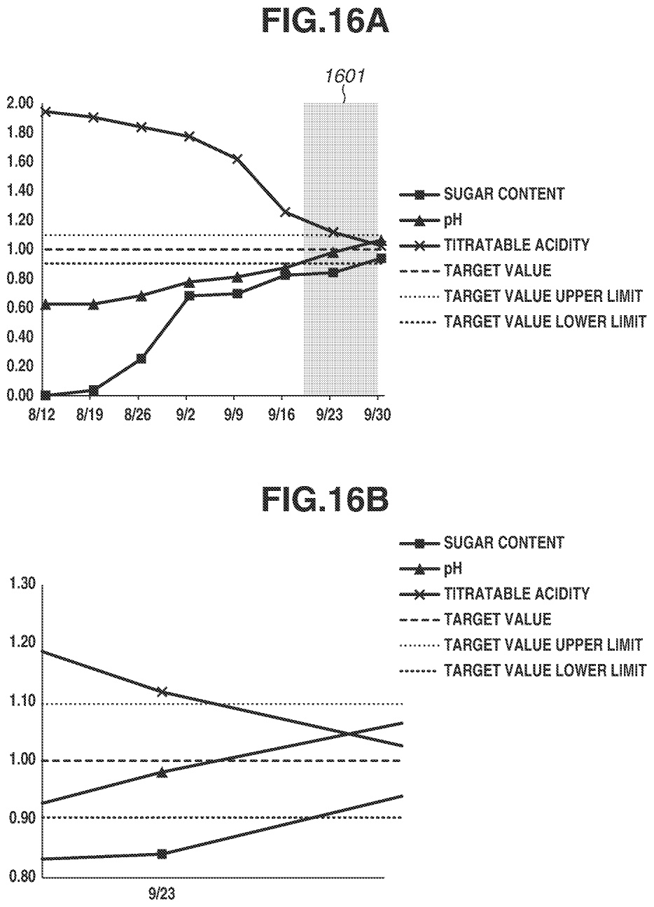

Furthermore, a configuration for enlarging a local area of the graph and displaying the enlarged local area may be further provided. This makes it possible to observe the changes in the measured values of the measurement items relative to the respective target values aligned at the same position so that a forecast is made well, even in a period in which the target values aligned at the same position, the tolerances, or the measured values of the respective measurement items are closely spaced. A modification in which the local area of the graph is enlarged and displayed will be described with reference to FIG. 16A and FIG. 16B.

FIG. 16A is a diagram illustrating the graph before an enlargement operation is performed. An enlargement period 1601 indicates a range of time (the horizontal axis) selected by a user operation as an enlargement display target. Hereinafter, the range of the time serving as the enlargement display target will be described as the enlargement period. The enlargement period 1601 may be input by an operation such as a drag operation on the graph, or may be input in a text field provided to accept the input of the enlargement period 1601. There may be adopted a configuration in which, after the input of the enlargement period 1601, enlargement processing is automatically executed on the graph, or a button for providing an instruction to execute the enlargement processing may be separately provided so that the enlargement processing can be executed any time.

The configuration in which the input of the enlargement period 1601 by the user is accepted has been described here, but in a case where the time when the measured value approaches the target value is known, there may be provided a configuration that enables the period to be enlarged by a simple operation. For example, in a graph for determining an expected harvest date based on a change in the degree of maturity of the crop, the measured value becomes closer to the target value toward the end of the growing period, and thus, reading the contents of the graph tends to be more difficult as the end of the growing period approaches. For this reason, a predetermined period from the end of the growing period is set as the enlargement period, so that a latter part of the growing period can be displayed in an enlarged manner by just pressing the button for providing an instruction to execute the enlargement processing. Alternatively, in a case where an instruction to generate the graph is provided after a predetermined period from the end of the growing period is designated, the graph in an enlarged state from the beginning may be generated.

FIG. 16B is a diagram illustrating a graph obtained by enlarging the graph in FIG. 16A based on the enlargement period 1601. FIG. 16B may be displayed in the same area as that in FIG. 16A, or may be displayed in an area different from that in FIG. 16A, on the display device 105. In addition, the range of the Y-axis may be adjusted based on the measured value included in the enlargement period 1601 and the measured value near that value when the enlargement processing is executed. A configuration in which the width of the tolerance can be adjusted after the tolerances of the respective measurement items are aligned may be further provided. This enables the user to make a decision while adjusting the width of the tolerance.

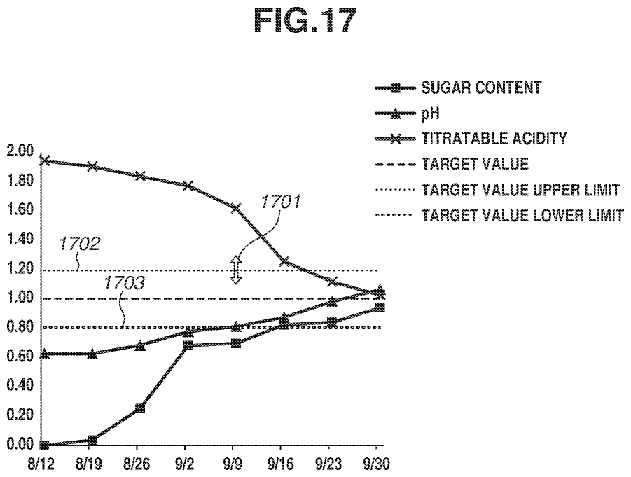

FIG. 17 is a diagram illustrating a graph including a configuration for directly adjusting the upper limit of the target value or the lower limit of the target value on the graph.

A cursor 1701 is a GUI component displayed by the display control unit 116 when an operation for dragging the upper limit of the target value or the lower limit of the target value is attempted with the input device 106. The user can adjust the upper limit or the lower limit of the target value on the graph by dragging an upper limit line 1702 or a lower limit line 1703. In response to an adjustment made to the upper limit of the target value, the same amount of change as the adjustment may be applied to the lower limit of the target value in the opposite direction. Conversely, in response to an adjustment made to the lower limit of the target value, the same amount of change as the adjustment may be applied to the upper limit of the target value in the opposite direction.

The example in which the width of the tolerance is directly adjusted with the input device 106 has been described here. However, in the second exemplary embodiment, the method of adjusting the tolerance on the graph is not limited to this example. For example, there may be adopted a configuration in which a text field for accepting the input of a numerical value as the allowable value is prepared and the width of the tolerance is adjust based on the allowable value input by the user. Alternatively, a GUI component for adding or subtracting a fixed numerical value to or from the current allowable value may be further displayed near the text field each time the text field is operated.

In this way, in the second exemplary embodiment, in a case where each of the measurement items has the tolerance of the target value, the widths and the positions of the tolerances of the target values of the respective measurement items are aligned on the graph. Even if the plurality of measurement items has ideally the correlation relationship concerning the growing of the crop, unevenness in the speed of gradually approaching the target value can occur depending on various circumstances in the actual site. For example, the item in which the measured value is likely to reach the target value early and the item in which the measured value is slow in reaching the target value can be mixed in the data of the plurality of measurement items simultaneously visualized. In such a case, for each of the plurality of measurement items varying to some extent in the period before reaching the target value, the user can check at a glance whether the measured value fits into the tolerance of the target value by looking at the graph visualized according to the second exemplary embodiment. In this way, in the second exemplary embodiment, making a decision using the graph can be effectively assisted. A modification in which a future measured value is predicted by a predetermined approximate function or a trained model and the result thereof is displayed on a graph as described in the first exemplary embodiment can also be applied to the second exemplary embodiment.

The present invention is not limited to the exemplary embodiments described above, and various modifications and changes can be made without departing from the spirit and the scope of the present invention. Therefore, to make the scope of the present invention public, the following claims are appended.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)TM), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An information processing apparatus configured to display results of measurement for a plurality of items about an agricultural crop as a graph, the information processing apparatus comprising:
a measurement information acquisition unit configured to acquire measurement information including an item for which the measurement is performed and a measured value obtained by the measurement;
a reference information acquisition unit configured to acquire information about a reference value set for each of the plurality of items, as a value to be compared with the measured value that changes over time;
a conversion unit configured to perform conversion of the measured value into a display coordinate value on a graph for each of the plurality of items based on the measurement information and the reference value, to make reference values set for the respective plurality of items agree with each other on the graph; and
a display control unit configured to execute control for displaying a graph including a plurality of pieces of series data corresponding to the respective plurality of items and having display coordinate values converted by the conversion unit, and an index indicating the reference values for the plurality of items.

2. The information processing apparatus according to claim 1, wherein the measurement for the plurality of items includes measurement of at least one of an acid level or a sugar content.

3. The information processing apparatus according to claim 2, wherein the measured value obtained by the measurement performed for each of the plurality of items about growing of the agricultural crop changes over time, and wherein the reference value is a target value set as a target with respect to the measured value of each of the plurality of items.

4. The information processing apparatus according to claim 3, wherein the target value is a value the measured value is expected to approach or reach over time.

5. The information processing apparatus according to claim 1, wherein the conversion unit performs the conversion by executing an operation varying depending on whether the measured value indicates an upward trend or a downward trend in the graph for each of the plurality of items.

6. The information processing apparatus according to claim 1,
wherein the measurement information acquisition unit stores the measurement information including the item for which the measurement is performed and the measured value obtained by the measurement in a storage unit, for measurement performed one or more times for any of the plurality of items, each time the measurement information is acquired, and
wherein the conversion unit performs the conversion by executing a common operation on one or more measured values for the same item of the measurement information stored in the storage unit.

7. The information processing apparatus according to claim 1,
wherein the measurement information further includes information about a date and time when the measurement is performed, and
wherein the graph is in a form indicating a change over time in a result of measurement repeatedly performed for each of the plurality of items about the agricultural crop .

8. The information processing apparatus according to claim 7,
wherein a horizontal axis of the graph is a temporal axis indicating the change over time in the result of the measurement repeatedly performed for each of the plurality of items about the agricultural crop , and
wherein the conversion unit performs the conversion to make the reference values set for the respective plurality of items agree with each other on the vertical axis of the graph.

9. The information processing apparatus according to claim 8, wherein the display control unit executes control for displaying an axis representing a range of measured values before being converted by the conversion unit of each of the plurality of items at a position corresponding to the axis of the graph.

10. The information processing apparatus according to claim 9, wherein the display control unit executes control for displaying the axis representing the range of the measured values before being converted by the conversion unit of each of the plurality of items to be switchable in response to a user operation.

11. The information processing apparatus according to claim 1, wherein the display control unit executes control for displaying the measured value before the conversion on the graph in response to a user operation specifying any of the measured values after the conversion plotted on the graph.

12. The information processing apparatus according to claim 8, wherein the display control unit further visualizes a date freely input by a user on the temporal axis of the graph.

13. The information processing apparatus according to claim 7,
wherein each of the plurality of items is an item for which a period for performing the measurement is set beforehand, and
wherein the display control unit displays the temporal axis of the graph to include at least a specific date of the set period.

14. The information processing apparatus according to claim 13, wherein the specific date of the set period is a date expected to be a last day of the period.

15. The information processing apparatus according to claim 13, further comprising a prediction unit configured to perform prediction about a change in a measured value obtained by measurement for each of the plurality of items,
wherein the display control unit displays a value predicted by the prediction unit on the graph, as a measured value in a period from a date of a latest measured value among measured values after the conversion plotted on the graph to the specific date.

16. The information processing apparatus according to claim 15, wherein the prediction unit performs the prediction by a trained model trained using a past measured value of each of the measurement items as training data.

17. The information processing apparatus according to claim 1, further comprising a condition acquisition unit configured to acquire a condition including a temporal range of measurement information to be used to generate the graph,
wherein the conversion unit performs the conversion of one or more measured values identified based on the condition of the measurement information stored in the storage unit.

18. The information processing apparatus according to claim 1,
wherein the reference information acquisition unit further acquires information about a tolerance set for each of the plurality of items,
wherein the conversion unit performs the conversion of the measured value into the display coordinate value on the graph for each of the plurality of items based on the measurement information, the reference value, and the information about the tolerance to make the reference values and tolerances set for the respective plurality of items agree with each other on the graph, and
wherein the display control unit executes control for displaying a graph including a plurality of pieces of series data corresponding to the respective plurality of items and having the display coordinate values converted by the conversion unit, and an index indicating the reference values and the tolerances for the plurality of items.

19. A control method of an information processing apparatus configured to display results of measurement for a plurality of items about an agricultural crop as a graph, the control method comprising:
acquiring measurement information including an item for which the measurement is performed and a measured value obtained by the measurement;
acquiring information about a reference value set for each of the plurality of items, as a value to be compared with the measured value that changes over time;
performing conversion of the measured value into a display coordinate value on a graph for each of the plurality of items based on the measurement information and the reference value, to make reference values set for the respective plurality of items agree with each other on the graph; and
executing control for displaying a graph including a plurality of pieces of series data corresponding to the respective plurality of items and having converted display coordinate values, and an index indicating the reference values for the plurality of items.

20. A storage medium storing a program for causing a computer to operate as an information processing apparatus configured to display results of measurement for a plurality of items about an agricultural crop as a graph, the program causing the information processing apparatus to execute:
acquiring measurement information including an item for which the measurement is performed and a measured value obtained by the measurement;
acquiring information about a reference value set for each of the plurality of items, as a value to be compared with the measured value that changes over time;
performing conversion of the measured value into a display coordinate value on a graph for each of the plurality of items based on the measurement information and the reference value, to make reference values set for the respective plurality of items agree with each other on the graph; and
executing control for displaying a graph including a plurality of pieces of series data corresponding to the respective plurality of items and having converted display coordinate values, and an index indicating the reference values for the plurality of items.
